(19) United States
(12) Patent Application Publication
(10) Pub. No.: US 2001/0016703 A1
(43) Pub. Date: Aug. 23, 2001

(54) System for reconstituting pastes and methods of using same

(76) Inventors: John Wironen, Alachua, FL (US); Penelope Kao, Alachua, FL (US); Andy Bernhardt, Alachua, FL (US)

Correspondence Address: Bencen & Van Dyke, 1630 Hillcrest Street, Orlando, FL, 32803, US

(21) Appl. No.: 09/751,929

(22) Filed: Dec. 29, 2000

Related U.S. Application Data
(63) Continuation-in-part of application No. 09/474,276, filed on Dec. 29, 1999.

(51) Int. Cl. A61M 37/00

(52) U.S. Cl. 604/89; 604/82; 604/191

(57) ABSTRACT

Disclosed herein is an efficient and cost-effective system for reconstituting bone pastes comprising a first syringe containing a reconstitution fluid and second syringe containing paste components, wherein said first and second syringes are communicatingly interconnectable. Also disclosed are novel methods for producing and storing bone pastes.

### BACKGROUND OF THE INVENTION

The ability to preserve biomedical substances has a great impact on the usefulness and applicability of such substances. This is especially true for important liquid or semi-solid substances whose vital components are prone to degradation and/ or spoiling when left in liquid form for extended periods of time. One method routinely utilized for preserving liquid or semi-solid substances involves the removal of the aqueous component of such substances (e.g. freeze-drying) to produce a dry powder. While converting liquid substances into powder form may address the problems of degradation and spoiling, problems still arise as to how to reconstitute such powders back into usable liquid form in a convenient and practical fashion.

An example of an increasingly important biomedical substance is osteogenic Bone Paste (WO 98/40113). Osteogenic Bone Paste has provided surgeons with a revolutionary means for repairing serious bone defects, as well as other bone-related injuries and problems. While current methods of utilizing bone paste have shown promise, there is a constant need for devising new, cost-effective techniques of storing and preserving bone paste and other biomedical substances, which, in turn, will increase their usefulness and adaptability to medical applications.

### SUMMARY OF THE INVENTION

The subject invention concerns a system that allows for a more expeditious and facile use and preparation of pastes. Specifically exemplified is a novel system for reconstituting bone paste, and/or other biomedical pastes or powders, that results in decreasing the time involved in processing such pastes, as well as diminishing the costs and inefficiencies associated with their storage. One aspect of the subject invention pertains to a system that comprises a first syringe containing reconstitution liquid and a second syringe containing paste components, wherein said first and second syringes are communicatingly interconnectable.

Another aspect of the subject invention pertains to a method of reconstituting a paste that comprises obtaining a first syringe that contains reconstitution liquid; obtaining a second syringe that contains paste components; and contacting (e.g. mixing) the contents of the first syringe with the contents of the second syringe.

A further aspect of the subject invention pertains to an article of manufacture comprising a syringe containing paste components under a vacuum.

Yet another aspect of the subject invention pertains to a kit comprising a first syringe containing reconstitution liquid, a second syringe containing paste components, and packaging materials.

Further still, another aspect of the subject invention pertains to a storing method for bone pastes that provides long-shelf life and simple implementation of the stored bone paste.

The subject invention provides an easy means for preparing pastes for utilization in medical and/or dental procedures. In addition, the subject invention cuts down on the costs of preserving bone and/or other biomedical pastes, and extends their shelf life. With respect to graft pastes, current methods require that they are stored at extremely cold temperatures (−80° C.). Such freezing presents a number of problems. Refrigeration at these temperatures is very costly, and the handling of the pastes at this temperature can be very time consuming. Also, extremely careful attention is required to ensure that the integrity of the paste is maintained. In contrast, the subject invention allows the graft paste to be processed down to its critical components, for example, by freeze-drying, and provides a simple means to reconstitute the paste components back into a workable paste, immediately before surgery. Processing the graft paste into its critical components allows for the storage of the paste at ambient (room) temperature for extended periods of time. These and other advantageous aspects of the subject invention are described in further detail below.

### DESCRIPTION OF THE DRAWINGS

FIG. 1 shows a side view of a disassembled apparatus for reconstituting pastes according to the principles of the subject invention, including a first syringe having a male connector end (FIG. 1A) and a second syringe having a female connector end (FIG. 1B).

FIG. 2 shows a longitudinal cross-section of the assembled apparatus as shown in FIG. 1.

FIG. 3 shows an embodiment of the invention illustrating an assembled syringe comprising a septum cap engaged thereon for accepting a needle.

FIG. 4 shows an embodiment of the invention illustrating the assembled syringe of FIG. 3 accepting a needle for the transfer of reconstitution fluid.

FIG. 5 shows a side view of an embodiment of the invention illustrating a syringe with a depression interlock disposed thereon for securing the syringe.

FIG. 6 shows a side view of an embodiment of the invention illustrating a syringe with a guard rack designed for aiding the loading of paste components into the syringe.

FIG. 7 shows a side view of an embodiment of the invention tailored for reconstituting larger quantities of paste components that comprises a three-way valve coupler.

FIG. 8 shows a side view of an embodiment of the invention comprising interconnectable syringes having larger bores and fasteners.

FIG. 9 shows a perspective view of the embodiment shown in FIG. 8.

FIG. 10 shows a side view 10A and a perspective view 10B of an embodiment of the invention pertaining to a plunger comprising an aperture and channel to facilitate expulsion of air from the contents of a syringe.

### DETAILED DISCLOSURE OF THE INVENTION

### Definitions

The term “communicatingly interconnectable” as used herein refers to the ability of two or more syringes to be connected in such as way as to allow the contents of a given syringe to be transferred to another syringe.

The term “paste” as used herein refers to a malleable composition useful in medical procedures. Pastes for use with the principles of the invention include, but are not limited to allograft pastes (e.g., osteogenic pastes or chondrogenic pastes), carrier associated Growth Factors, carrier associated mineralized particles, morsellized skin or other tissue, Fibrin powder, Fibrin/plasminogen glue, biomedical plastics, Demineralized Bone Matrix (DBM)/glycerol, cortico cancellous chips (CCC), DBM/pleuronic F127, and DBM/CCC/F127, human tissue/polyesters or polyhydroxy compounds, or polyvinyl compounds or polyamino compounds or polycarbonate compounds or any other suitable viscous carrier. Preferably, pastes used in accordance with the principles of the subject invention are graft pastes having osteogenic or chondrogenic properties.

In a most preferred embodiment, the paste for use as taught herein contains a carrier, an osteoconductive component, and an osteoinductive component. Carriers can include, but are not limited to, gelatin, collagen, glycerol, and/or mixtures thereof. Osteoconductive materials suitable for use with the subject invention include, but are not limited to, hydroxapatite (HA), tricalcium phosphate (TCP), CCC, bioactive glass, bioactive ceramics, and/or mixtures thereof. Osteoinductive materials suitable for use with the subject invention include, but are not limited to, DBM, bone morphogenic protein (BMP), TGF-beta, PDGF, and/or mixtures thereof.

The term “paste components” as used herein refers to those components of a paste that are produced by removing liquid from a paste, and are capable of reconstitution into a workable paste upon contact with a reconstitution liquid. One skilled in the art will readily appreciate processing methods suitable in accord with the principles of the subject invention. Preferably, paste components are those components produced by removing water from a paste of interest, such as, but not limited to, by freeze-drying of a paste.

The term “reconstitution liquid” as used herein refers to a liquid capable of reconstituting paste components into a workable paste upon mixing with the paste components. Reconstitution liquids useful in accordance with the principles of the subject invention include, but are not limited to, water and water-based salines, or any other non-toxic fluid such as blood, Growth Factor solutions, antibiotic solutions, protein solutions, gelatin solutions, analgesic solutions, and platelet rich plasma. In a preferred embodiment, the reconstitution fluid is blood, or fractions thereof (e.g., serum or plasma). More preferably, paste components are reconstituted with a patient's blood to form a paste that is injected back into the patient.

The term “syringe” as used herein refers to an apparatus that is capable of containing a substance, connecting with another apparatus, and transferring that substance to that other apparatus. In a preferred embodiment, two or more syringes are connectable with each other, and are capable of sending and receiving their contents to and from each other. The mode of actuating the transfer of a syringe's contents can be by hand, but can include other mechanical means, for example, by a motor. In an even more preferred embodiment, the syringe is capable of holding its contents under a vacuum, preferably, up to 5 years or more. Further, as discussed above, the syringes are preferably communicatingly interconnectable. One means of connection includes, but is not limited to, the presence of a male Luer-type connector on a first syringe and a female Luer-type connector on a second syringe. The presence of this reciprocal male and female connection not only provides for a simple and efficient interconnection, but decreases undesirable “dead” space between the two syringes. Other means of connection will be readily appreciated by those skilled in the art, such as, for example, a stop-cock for receiving two male connectors or a two-sided female adapter for receiving two male connectors.

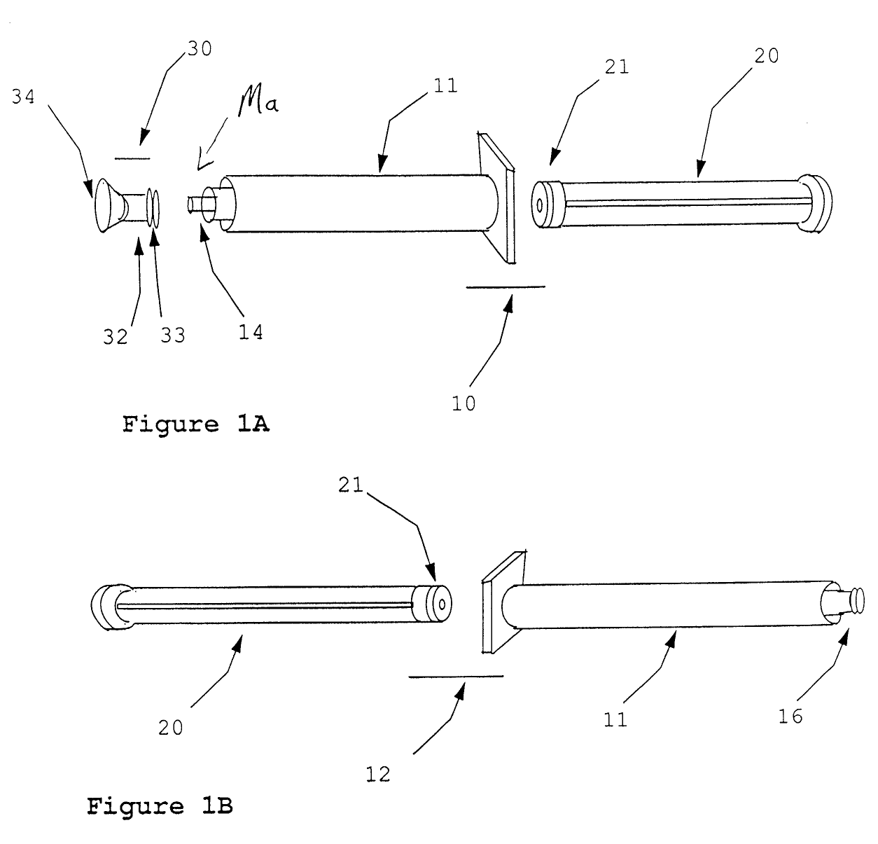

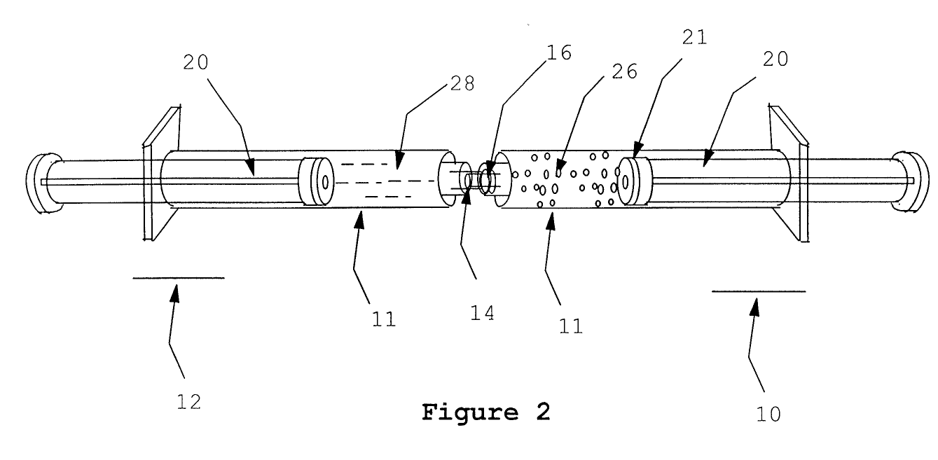

Turning now to the drawings, a reconstitution system is shown generally in FIGS. 1-3. The system comprises a first syringe 10 and a second syringe 12 communicatingly interconnectable with each other. Syringes 10 and 12 comprise substantially cylindrical body portions 11, suitable for receiving a plunger rod 20. Plunger rod 20 has disposed on one end a crown 21, made of a material for forming a seal between its surface and the surface of the inner walls of the syringe body 11. Materials contemplated for the crown 21 include, but are not limited to, rubber and plastic. Second syringe 12 preferably has a female end 16 for receiving a male end 14 of first syringe 10. As shown in FIG. 2, the first syringe 10 comprises paste components 26 and the second syringe 12 comprises reconstitution fluid 28. The reciprocating male and female ends provide for a tightly sealed connection that minimizes “dead” space between the two syringes, thereby alleviating unwanted air bubbles. Alternatively, albeit less preferred, a first syringe and a second syringe may be connected by an adapter having two male ends, two female ends or a male end and female end, depending on the ends of the first and second syringes. Further, the first syringe and second syringe may be connected through friction by snapping into each other, or snapping into an adapter.

In yet another embodiment, the introduction of air is minimized in the system through the use of a stopcock valve. In this embodiment, a first syringe and a second syringe are evacuated and then communicatingly interconnected via a stopcock valve. Upon rotation of the stopcock valve, the contents of one syringe are allowed to flow into the other syringe. In light of the teachings herein, those skilled in the art will appreciate the types of valves suitable for this purpose. The important aspect of the valve is the ability to interconnect to at least two syringes.

Operation of the system to reconstitute a paste can comprise applying pressure to a first plunger rod 20 of syringe 12 which thereby pushes the reconstitution fluid 28 into syringe 10. Upon transfer of the reconstitution fluid 28, it is brought into contact with the paste components 26. Preferably, the plunger rod 20 in syringe 10 is gradually pulled in coordination with the pushing of rod 20 in syringe 12 to create negative pressure and more space in syringe 10 to aid in and accommodate the transfer of the reconstitution fluid 28. In a preferred embodiment, the paste components are inserted into syringe 10, and syringe 10 is evacuated such that the paste components are held under a vacuum. Once an adequate amount of reconstitution fluid is transferred from syringe 12 to syringe 10, the contents of syringe 10 can be transferred back to syringe 12 whereby the paste components and reconstitution fluid are mixed resulting in a useable paste. Preferably, the contents of each syringe are transferred several times until the desired consistency of the paste is achieved.

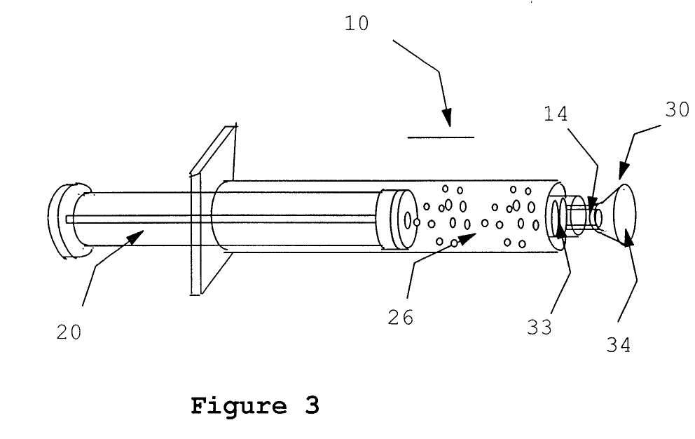

As shown in FIG. 3, another embodiment of the invention is directed to a system for reconstituting pastes comprising a first syringe 10 containing paste components 26 held under a vacuum and a removable cap 30. The removable cap 30 comprises a rigid portion 33 that is engaged to the end of the syringe 10 and a septum portion 34. The rigid portion preferably includes an end 33 configured to be removably engageable to the end of a syringe. More preferably, the end 33 is a male or female connecting end. The septum portion is preferably made of a material that is capable of accepting an injection means (for example, a needle; see U.S. Pat. No. 5,951,160 for other examples of injection means) while still maintaining the seal of the syringe 10, such as, but not limited to, rubber, silicone, plastic and other elastic materials.

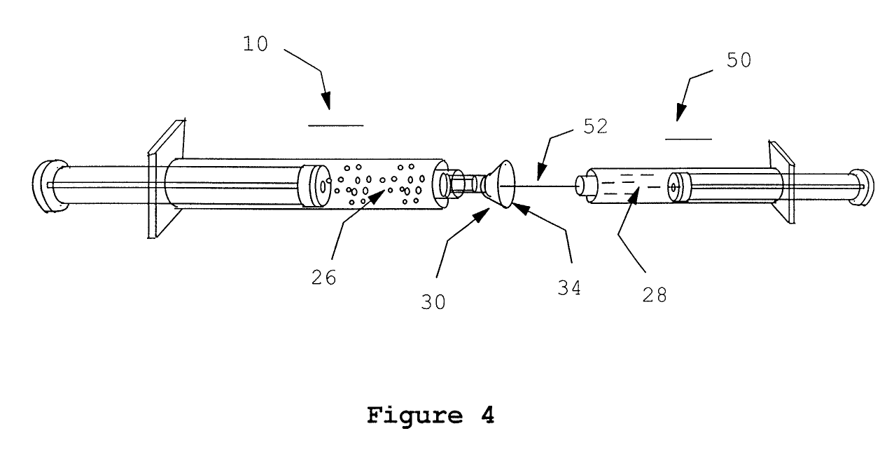

A further embodiment shown in FIG. 4 pertains to a system for reconstituting pastes. Operation of this embodiment involves drawing reconstitution fluid into a syringe 50 equipped with a needle 52 on its end. The needle 52 is inserted into syringe 10 through the septum portion 34 of the cap 30 and the reconstitution fluid 28 is transferred into the syringe 10. Preferably, the reconstitution fluid 28 is transferred while the transfer of any air is avoided. Once the reconstitution fluid 28 is transferred into syringe 10, the cap 30 is removed, at which time a second syringe is interconnected with syringe 10. The paste components and reconstitution fluid present in syringe 10 are mixed by transfer back and forth from syringe 10 to the second syringe until a paste of a desired consistency is formed. In a preferred embodiment, the paste components comprise a gelatin material which is melted by heating prior to mixing. Preferably, heating may occur in a water bath for 3 to 5 minutes. After the paste is formed, the syringe 10 and the second syringe are dissociated, and the paste is ejected as needed.

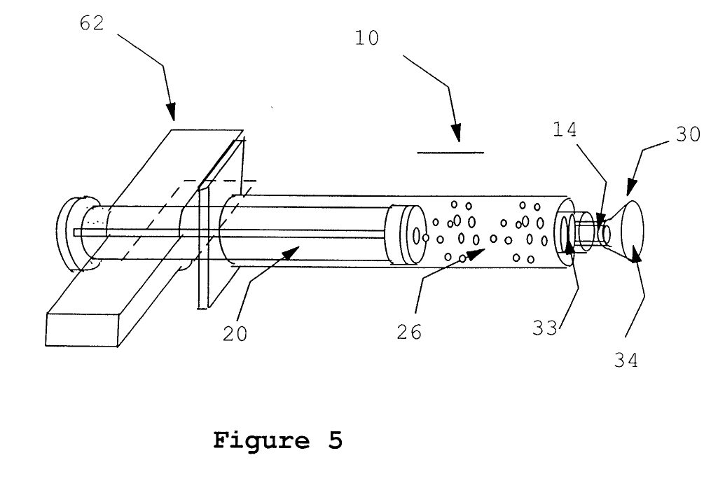

In another embodiment, as shown in FIG. 5, the subject invention is directed to an article of manufacture that comprises a syringe 10 that contains paste components 26 being held under a vacuum. To aid in preventing the inadvertent release of the vacuum, a depression interlock 62 is provided that protects the plunger rod 18 from sliding. Alternatively, the syringe comprises a cap 30 engaged at one end. The cap 30 preferably comprises a rigid portion 33 and a septum portion 34. Those skilled in the art will readily appreciate, in light of the teachings herein, other devices suitable for preventing the inadvertent depression of the plunger rod.

A further embodiment of the subject invention is directed to kit comprising a first syringe containing paste components, a second syringe, and a container for housing the syringes. Preferably, the second syringe contains reconstitution fluid. In a more preferred embodiment, the kit comprises a cap that has a rigid portion for engaging a syringe and a septum for accepting a needle. In an even more preferred embodiment, the kit comprises a needle having an end for engaging a syringe. Alternatively,

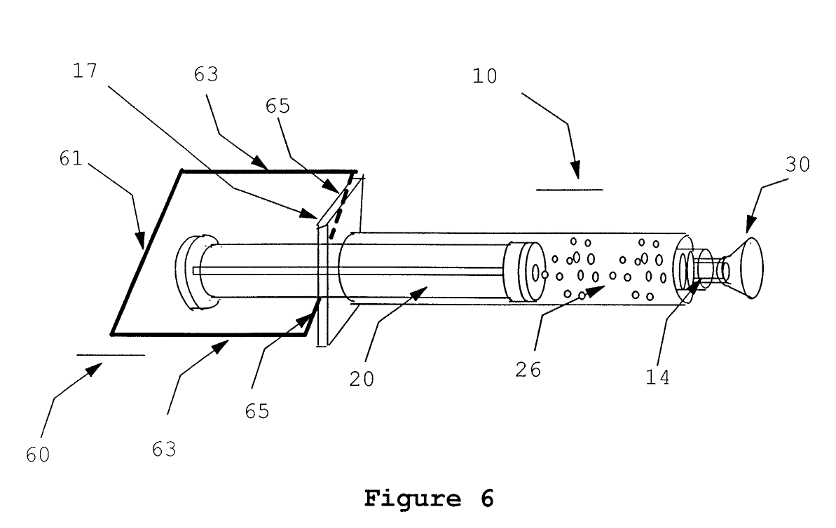

FIG. 6 shows an embodiment of the invention that is directed to a process for packing paste components into a syringe. Paste or paste components 26 are placed within a syringe 10 having disposed thereon a guard rack 60 having a bottom 61, two or more sides 63, and two or more top portions 65 extending perpendicularly from the ends of the sides 63 that are opposite the bottom 61. The top portions 65 preferably extend toward each other such that a space is formed between the two extended top portions 65 that is of a suitable size to accommodate a plunger rod 20 and support the bottom end 17 of the syringe 10. To produce an evacuated syringe, a cap 30 is placed loosely onto the top end 14 of the syringe 10, and the syringe 10 disposed on the guard rack 60 is placed in a lyophilizer. Upon lyophilization of the contents in the syringe 10, the syringe 10 and guard rack 60 is raised such that the cap 30 comes into contact with a roof surface of the lyophilizer. The cap 30 is contacted with a force sufficient to firmly engage the cap 30 onto the syringe 10, to thereby form and maintain a vacuum. Alternatively, a valve is removably engaged to said syringe. The configuration of the guard rack 60 and the syringe 10 prevents the depression of the plunger rod during loading of the syringe 10. Those skilled in the art will readily appreciate, in view of the teachings herein, other devices suitable for preventing depression of the plunger rod such as the depression interlock discussed above.

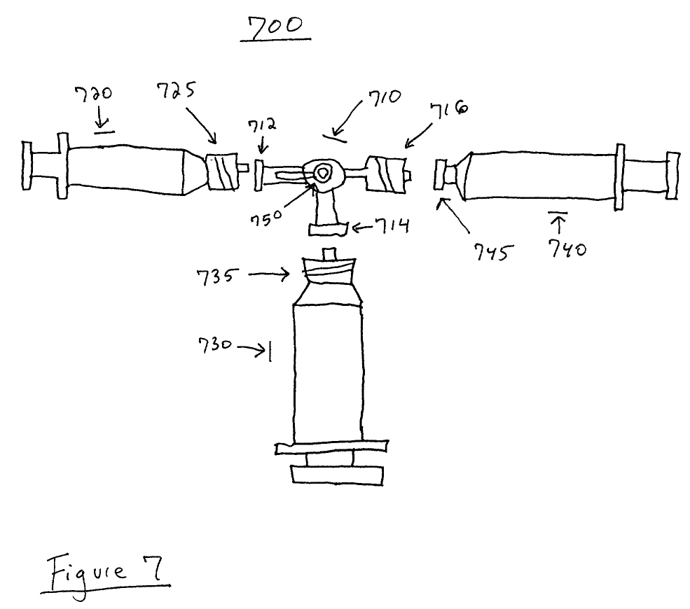

FIG. 7 shows a further embodiment 700 of the invention that is especially tailored to reconstitute larger quantities of paste components. Embodiment 700 comprises a three-way valve coupler 710 that has three Luer-lok adapter ends: a first female end 712, a second female end 714, and a male end 716 that are interconnectable with corresponding Luer-lok ends on a first syringe 720 having a male end 725, a second syringe 730 having a male end 735, and a third syringe 740 having a female end 745, respectively. Those skilled in the art will recognize that the Luer-lok adapter ends of the three-way valve coupler 710 are readily interchangeable with either male or female Luer-lok ends. The three-way valve coupler 710 is equipped with a rotatable valve 750 (preferably a conventional Qosina valve) that is capable of directing communication between two of three adapter ends. In a preferred embodiment, first syringe 720 and third syringe 740 are 5 cc syringes and second syringe 730 is a 20 cc syringe.

The preferred operation of embodiment 700 is as follows: Syringes 720, 730, and 740 are connected to the three-way valve coupler 710. Syringe 720 contains reconstitution fluid, syringe 740 contain paste components, and syringe 730 is empty. Rotatable valve 750 is turned to 9 o'clock (as shown) to close flow to syringe 720 and opening flow between syringes 730 and 740. The plunger 755 of syringe 730 is pulled to draw air out of paste components contained in syringe 740. After removing air, syringe 730 can be removed and rotatable valve 750 is turned to open flow between syringes 720 and 740. The plunger 760 of syringe 720 is pushed and the plunger 765 of syringe 740 is pulled to draw reconstitution fluid into syringe 740. The contents of syringe 740 is then transferred back to syringe 720 and back to syringe 740, and repeated if necessary, to mix the reconstitution fluid with the paste components, until desired mixture is achieved.

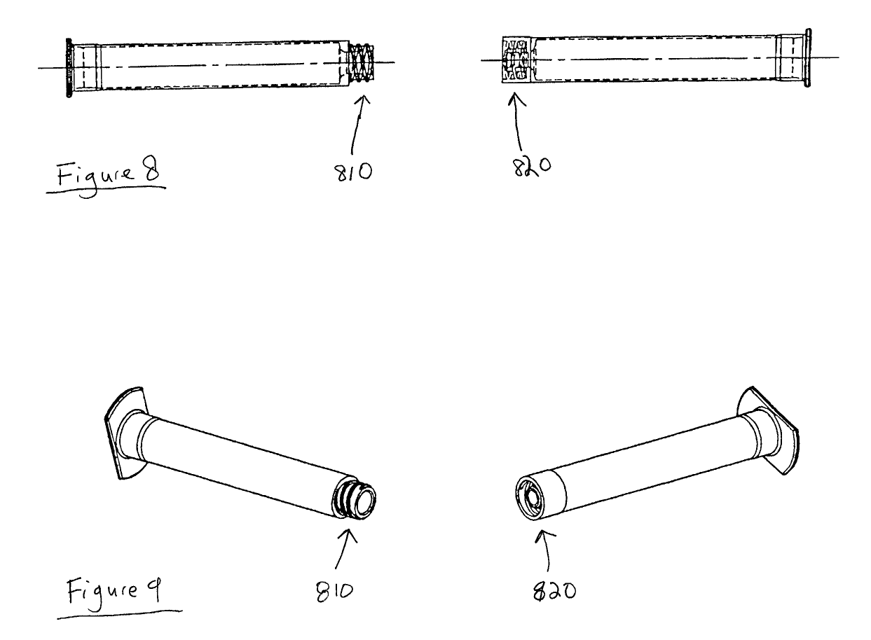

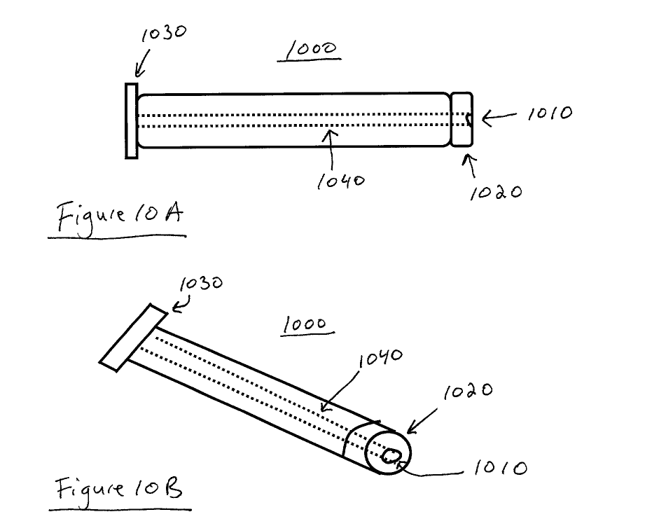

Alternatively, or preferably, when reconstituting larger quantities of paste components, syringes equipped with larger bores over standard Luer-lok ends are used to accommodate and facilitate flow of the materials to and from the syringes. FIGS. 8 and 9 represent a side view and perspective view, respectively, of syringes equipped with larger bores: female 810 and male 820. Those skilled in the art will appreciate that the ends of the syringes and three-way valve coupler shown in FIG. 7 and described above for embodiment 700, as well as the other connecting ends of devices described herein, can be substituted with ends having extra-large bores. Preferably, the extra-large bores range from about 0.4 inches to about 0.6 inches in diameter for 5-10 cc syringes.

Typically, it is desirous to remove air from the paste components before, during or after reconstitution is conducted. In a specific embodiment, the removal of air is facilitated by providing an aperture in the plunger of the syringes used in accord with the teachings herein. For example, FIG. 10 shows a plunger 1000 having an aperture 1010 at its end 1020 in contact with paste components, wherein the aperture communicates with the other end of the plunger 1030 through a channel 1040 defined within the plunger 1000.

Preferably, to prevent escape of paste components or reconstitution fluid, the aperture is covered with an air-permeable membrane or filled with an air-permeable plug. Materials for the plug, membrane or other similar structures are commercially available and include, for example, TF Membrane Filters, Gelman (VWR Scientific) or Porex Hydrophobic vents (Porex Corp.). According to the principles of this embodiment, as the plunger is pushed against the contents of the syringe, the pressure caused thereby acts to push the air contained in the contents through the aperture and expelled out of the syringe.

### EXAMPLE 1

Syringe A is a male Luer-lock. Syringe B is a female Luer-lock having a septum cap disposed thereon and paste components contained therein.

(1) Attach a 22-30 gauge needle to Syringe A.

(2) Draw up an appropriate amount of reconstitution fluid into syringe A, preferably blood or plasma.

(3) Plunge needle through the septum cap on syringe B and inject the reconstitution into syringe B.

(4) Warm Syringe B for 2-6 minutes in 49 degree Celsius water bath.

(5) Remove the septum cap from Syringe B. Remove the needle from syringe A.

(6) Attach Syringe A to Syringe B.

(7) Transfer the contents from Syringe A to Syringe B.

(8) Transfer the contents from Syringe B to Syringe A.

(9) Repeat steps (7) and (8) until reconstitution fluid and paste components are mixed to form a paste of a desired consistency.

(10) Use or rewarm the paste.

The teachings of all patents and publications cited throughout this specification are incorporated by reference in their entirety to the extent not inconsistent with the teachings herein.

It should be understood that the examples and embodiments described herein are for illustrative purposes only and that various modifications or changes in light thereof will be suggested to persons skilled in the art and are to be included within the spirit and purview of this application and the scope of the appended claims.

What is claimed is:

1. A system for reconstituting a paste comprising:
a) a first syringe containing a reconstitution liquid; and
b) a second syringe containing paste components, wherein said first syringe and said second syringe are communicatingly interconnectable.

2. The system of claim 1 wherein said paste components are held under a vacuum.

3. The system of claim 2 further comprising packaging to house said second syringe, wherein said packaging is evacuated to thereby create said vacuum.

4. The system of claim 1 wherein said second syringe is susceptible to contain said paste components under a vacuum.

5. The system of claim 1 wherein said second syringe comprises a septum cap disposed on the end of said syringe, wherein said septum cap is suitable for receiving an injection means.

6. The system of claim 5 wherein said second syringe is under a vacuum and wherein said septum cap is capable of maintaining said vacuum upon receiving said injection means.

7. The system of claim 1 wherein said paste components comprise one or more components selected from the group of consisting of osteogenic pastes, chondrogenic pastes, carrier associated Growth Factors, carrier associated mineralized particles, morsellized skin or other tissue, Fibrin powder, Fibrin/plasminogen glue, bioactive glass, bioactive ceramic, Demineralized Bone Matrix (DBM)/glycerol, cortico cancellous chips (CCC), DBM/pleuronic F127, DBM/CCC/F127, and human tissue/polyesters or polyhydroxy compounds, or polyvinyl compounds or polyamino compounds or polycarbonate compounds.

8. The system of claim 1 wherein said reconstitution fluid is selected from the group consisting water, water-based salines, blood or fractions thereof, protein solutions, gelatin solutions, growth factor solutions, antibiotic solutions, analgesic solutions, platelet rich plasma, and mixtures thereof.

9. The system of claim 8 wherein said reconstitution fluid is blood or fractions thereof.

10. The system of claim 1 wherein said paste components comprise a carrier component, an osteoconductive component, and an osteoinductive component.

11. A method of forming a paste comprising:
a) obtaining a first syringe containing a reconstitution liquid;
b) obtaining a second syringe containing paste components;
c) interconnecting said first and second syringes;
d) contacting the contents of said first syringe with the contents of said second syringe; and
e) transferring said reconstitution liquid and said paste components from said first syringe or said second syringe to the opposite syringe at least once, thereby mixing said reconstitution liquid and paste components into a workable paste.

12. The method of claim 11 wherein said paste components comprise one or more components selected from the group of consisting of osteogenic paste components, chondrogenic paste components, carrier associated Growth Factors, carrier associated mineralized particles, morsellized skin or other tissue, Fibrin powder, Fibrin/plasminogen glue, bioactive glass, bioactive ceramic, Demineralized Bone Matrix (DBM)/glycerol, cortico cancellous chips (CCC), DBM/pleuronic F127, DBM/CCC/F127, human tissue/polyesters or polyhydroxy compounds, or polyvinyl compounds or polyamino compounds or polycarbonate compounds, DBM and gelatin.

13. The method of claim 11 wherein said reconstitution fluid is selected from the group consisting water, water-based salines, blood or fractions thereof, protein solutions, gelatin solutions, growth factor solutions, antibiotic solutions, analgesic solutions, platelet rich plasma, and mixtures thereof.

14. The method of claim 13 wherein said reconstitution fluid is blood or fractions thereof.

15. The method of claim 11 wherein said paste components comprise a carrier component, an osteoconductive component, and an osteoinductive component.

16. The method of claim 11 further comprising the step of heating said reconstitution liquid, said paste components, or both.

17. A method of storing paste comprising:
a) drying said paste to produce paste components;
b) inserting paste components into one or more syringes; and
c) evacuating said one or more syringes such that said paste components are held under a vacuum.

18. The method of claim 17 further comprising the step of storing said evacuated one or more syringes for at least 24 hours.

19. The method of claim 17 further comprising sealing said one or more syringes with a septum cap.

20. An article of manufacture comprising a syringe containing paste components held under a vacuum.

21. The article of manufacture of claim 20 further comprising a septum cap removably engaged to said syringe.

22. The article of manufacture of claim 20 further comprising a depression interlock disposed on said syringe.

23. The article of manufacture of claim 20 further comprising packaging for housing said syringe, wherein said packaging is evacuated to thereby form said vacuum.

24. The article of manufacture of claim 20 further comprising a valve removably engaged to said syringe.

25. A kit for producing a graft paste comprising:
a) a first syringe containing a reconstitution liquid;
b) a second syringe containing paste components, wherein said first syringe and said second syringe are communicatingly interconnectable and are susceptible to holding their contents under a vacuum; and
c) a container for housing said first and second syringes.

26. A method of loading a syringe to contain paste components comprising:
a) subjecting a syringe containing paste components to a vacuum environment; and
b) sealing said syringe while said syringe is in said vacuum environment.

27. The method of claim 26 wherein said sealing said syringe comprises pushing a cap onto an open end of said syringe.

28. The method of claim 27 wherein said cap has a septum portion thereon.

29. A cap designed for vacuum sealing of a syringe, said cap comprising an end configured to removably engage into a syringe, and a septum portion suitable for accepting an injection means.

30. A method of administering a paste to a patient comprising:
(a) obtaining a syringe comprising paste components;
(b) drawing a body fluid from said patient;
(c) reconstituting said paste components into a paste using said body fluid; and
(d) injecting said paste into said patient.

31. The method of claim 30 wherein said body fluid is blood, interstitial fluid or fractions thereof.

32. A method of forming a paste comprising:
(a) obtaining a first syringe containing paste components, wherein a septum cap is engaged to one end of said first syringe and wherein said first syringe is evacuated;
(b) injecting a reconstitution fluid into said first syringe via said septum cap;
(c) interconnecting a second syringe to said first syringe;
(d) transferring said reconstitution liquid and said paste components from said first syringe to said second syringe at least once, thereby mixing said reconstitution liquid and paste components into a workable paste.

33. The system of claim 1 wherein at least one of said first syringe and said second syringe comprises a plunger, said plunger comprising an aperture defined on a first end, an elongated portion, and a second end oppositely positioned to said first end; wherein said elongate portion comprises a channel defined therein such that air can travel from said first end to said second end through said channel.

34. The system of claim 33, wherein said aperture is covered by a gas-permeable membrane.

35. The system of claim 33, wherein said aperture is filled with a gas-permeable plug.

36. The system of claim 1 further comprising a third syringe which is empty and a coupler device comprising a valve; wherein said first syringe, said second syringe, and said third syringe are communicatingly interconnectable with each other via interconnection with said coupler device.

37. A method of forming a paste using the system of claim 36 comprising the steps of:
interconnecting said first syringe, said second syringe, and said third syringe to said coupler device;
opening said valve such that said second syringe and said third syringe can communicate; and
drawing air out of said second syringe.

38. The method of claim 11, further comprising removing air from said first syringe or second syringe, or both.
